(19) United States
(12) Patent Application Publication
(10) Pub. No.: US 2008/0226572 A1
(43) Pub. Date: Sep. 18, 2008

(54) Cosmetic emulsion containing silicone compounds

(75) Inventor: Guillaume Cassin, Villebon Sur Yvette (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 12/003,016

(22) Filed: Dec. 19, 2007

Related U.S. Application Data
(60) Provisional application No. 60/883,173, filed on Jan. 3, 2007.

(30) Foreign Application Priority Data
Dec. 20, 2006 (FR) 06 55705

(51) Int. Cl. A61K 8/72; A61K 8/06; A61Q 1/06; A61Q 3/02; A61Q 5/00

(52) U.S. Cl. 424/61; 424/78.03; 424/70.12; 424/64

(57) ABSTRACT

The present invention mainly relates to a cosmetic kit for make-up or non therapeutic care of keratinous substance(s) comprising at least two compositions and containing at least one compound X and at least one compound Y, compounds X and Y being capable of reacting together and at least one of the compounds being a silicone compound, with at least one of the compositions being an emulsion.

This non provisional application claims the benefit of French Application No. 06 55705 filed on Dec. 20, 2006 and U.S. Provisional Application No. 60/883,173 filed on Jan. 3, 2007.

The present invention mainly relates to a cosmetic kit for make-up or non therapeutic care of keratinous substance(s), comprising at least two compositions and containing at least one compound X and at least one compound Y, compounds X and Y being capable of reacting together if necessary in the presence of a catalyst or of a peroxide, and at least one of the compounds X or Y being a silicone compound, with at least one of the compositions being an emulsion.

The kits according to the invention can be products for make-up or care of keratinous substances, in particular the skin, the lips, the eyelashes, the eyebrows or the nails.

More specifically, the make-up products may be of the type such as foundations, make-up rouges, eyeshadows, concealer products, blushers, lipsticks, lip balms, lip glosses, mascaras, eyeliners, body make-up products or skin colouring products.

The skincare products may be a composition for protecting, treating or caring for the face, the hands, the feet, the major anatomical folds or the body (for example a day cream, night cream, make-up-removing cream, antisun composition, protective or care body milk, after-sun milk, skincare lotion, gel or mousse, artificial tanning composition); an aftershave composition.

The present invention is more particularly directed toward proposing a novel method for formulating cosmetic compositions, making it possible to obtain a film deposited on the keratin materials that has good cosmetic properties, especially in terms of staying power and matting effect, and that forms a comfortable deposit on the skin.

Recently, the inventors have found that it is possible to obtain such properties by exploiting the capacity of certain compounds, especially silicone compounds, to interact when they are placed in contact if necessary in the presence of a catalyst or of a peroxide and to constitute, after their interaction, a polymer film.

Thus, compounds referred to as compound X and compound Y, as defined below, prove to be capable of polymerizing in situ, at atmospheric pressure and room temperature, and of forming films that are advantageously biocompatible, non-tacky and slightly opalescent, or even peelable. Such systems are notably described partly in patents WO 01/96450 and GB 2 407 496 from Dow Corning.

These polymer films, which may be formed in situ on a support, especially of keratin material type, are found to have advantageous properties in cosmetic terms, i.e. good adhesion, good staying power and satisfactory comfort.

Generally these films also have a certain degree of gloss. Now, although these properties of gloss are required for certain cosmetic products such as lipsticks and nail varnishes, they may conversely be undesirable for other cosmetic products such as foundations and care products. Consequently, the present invention aims more particularly to propose cosmetic products for make-up and/or care that are capable of forming a film in situ by placing the abovementioned compounds in contact and that, on the other hand, are without any gloss effect.

The inventors discovered that it is possible to obtain the abovementioned properties provided at least one of the compounds X and Y is formulated in the oily phase of a simple or multiple W/O/W emulsion.

Simple emulsion is understood to means an oil-in-water (OMW) or water-in-oil (W/O) emulsion.

Thus, according to a first aspect, the present invention relates to a cosmetic kit for make-up or non therapeutic care of keratinous substance(s) comprising at least two compositions that are different and are packaged separately, the kit comprising at least one compound X, at least one compound Y, and optionally at least one catalyst or a peroxide, with at least one of compounds X or Y being a silicone compound and said compounds X and Y being capable of reacting together by a hydrosilylation reaction in the presence of a catalyst, or by a condensation reaction, or by a crosslinking reaction in the presence of a peroxide, when they are brought into contact with one another, said kit being such that compounds X and Y and the catalyst or the peroxide, when they are present, are not present simultaneously in the same composition and that at least one of the compositions of said kit is in the form of a simple or multiple W/O/W emulsion in which said compound X or Y is present in the oily phase.

The compound(s) X and the compound(s) Y may be applied to the keratin materials using several compositions containing the compound(s) X and the compound(s) Y, alone or as a mixture, or using a single composition containing the compound(s) X and the compound(s) Y.

According to a first variant embodiment, said kit comprises at least
i. a first composition containing in a physiologically acceptable medium at least one compound X and
ii. a second composition containing in a physiologically acceptable medium at least one compound Y,

with at least one of said first and second compositions being in the form of a simple or multiple W/O/W emulsion in which the compounds X or Y is present in the oily phase, and with at least one of said first and second compositions additionally containing if necessary at least one catalyst or one peroxide.

The first and second compositions are different from each other.

For example, the first composition is advantageously free of compound Y and the second composition is advantageously free of compound X. In point of fact, with regard to their great reactivity toward each other, compounds X and Y are not simultaneously present in a first and/or second composition forming a kit according to the invention when their interaction is not conditioned by the presence of a catalyst or of peroxide.

According to a first embodiment, said kit may contain only one of the two compositions in the form of an emulsion.

According to a second embodiment, the two compositions are in the form of a simple or W/O/W multiple emulsion.

According to a second embodiment variant, said kit comprises at least:
i. a first composition containing, in a physiologically acceptable medium, at least one compound X and one compound Y, said compounds X and Y being capable of reacting together in the presence of a catalyst via a hydrosilylation reaction, or via a condensation reaction, or via a crosslinking reaction in the presence of a peroxide, when they are placed in contact with each other, and
ii. a second composition containing, in a physiologically acceptable medium, at least said catalyst or said peroxide necessary for the interaction of compounds X and Y,
   with said first composition being in the form of a simple or multiple (W/O/W) emulsion in which compounds X and Y are present in the oily phase.

In particular, the kit in accordance with the invention may comprise at least one catalyst.

In particular, said catalyst is formulated in the aqueous phase.

Advantageously, the composition(s) in the form of an emulsion in the kits according to the invention is (are) oil-in-water direct emulsions.

Unexpectedly, the film obtained after applying the compositions forming the kit according to the invention is found to be without any gloss effect.

Thus, this film may be characterized by a matting parameter featured by the symbol R, the value of which is less than 1.2 when it is determined according to the protocol described below.

Preferably, the compositions of the kit and in particular the first composition that includes compound X and the second composition that includes compound Y of the kit are packaged in separate packaging.

For example, each composition can be packaged separately in the same packaging article, for example in a two-compartment pen, the base composition being delivered by one end of the pen and the top composition being delivered by the other end of the pen, each end being closed notably hermetically with a cap. Each composition can also be packaged in a compartment within the same packaging article, the two compositions being mixed at the end or ends of the packaging article during delivery of each composition.

Alternatively, each of the first and second compositions can be packaged in a different packaging article.

In the sense of the invention, notably in the embodiment where the composition is obtained as described above, namely by mixing, at the time of use, a first composition containing at least compound X and a second composition containing at least compound Y, it is to be understood that the mixture thus formed comprises compounds X and/or Y in a form that has not yet reacted and not exclusively in the form of their reaction product by hydrosilylation, by polycondensation and/or by crosslinking in the presence of a peroxide.

Thus, formation of the reaction product according to the invention can either be carried out directly on the surface of the keratinous substance that is to be treated, or initiated just before application by extemporaneous mixing of compounds X and Y in conditions favourable for their interaction, formation of the reaction product being in the latter case finalized on the surface of the keratinous substance.

For obvious reasons, and in view of the great reactivity of compounds X and/or Y, it is in fact necessary that their application should be carried out in conditions that are favourable for the manageability of the composition containing it (or them) notably with respect to its spreading, for example. The method according to the invention therefore employs a composition containing compounds X and Y, and therefore not congealed in the form of the expected final film resulting from reaction of all of X and/or of all of Y.

The invention also relates to a method of cosmetic care and/or make-up of keratinous substance(s) comprising at least the application in the form of at least one simple or multiple (W/O/W) emulsion (a), of one or more compounds X (b), of one or more compounds Y, with at least one of compounds X and Y being a silicone compound and said compounds X and Y being capable of reacting together by a hydrosilylation reaction in the presence of a catalyst, or by a condensation reaction, or by a crosslinking reaction in the presence of a peroxide, when they are brought into contact with one another, and if necessary (c) of a catalyst or a peroxide if needed for the interaction of said compound X with said compound Y, and applications (a), (b) and (c) can be simultaneous or consecutive in any order provided that it promotes the interaction of said compounds X and Y.

Thus, the compound or compounds X, the compound or compounds Y, can be applied on keratinous substances from several compositions, the compositions containing respectively the compound or compounds X, the compound or compounds Y, and if applicable additionally at least one catalyst or one peroxide, on their own or mixed, or from a single composition containing the compound or compounds X and the compound or compounds Y and if necessary additionally at least one catalyst or one peroxide.

According to a particular embodiment of the invention, one apply, according to a cosmetic method for make-up and/or care of keratinous substances, notably of human skin, on said keratinous substances at least one layer of a first composition containing in a physiologically acceptable medium at least one compound X and at least one layer of a second composition containing in a physiologically acceptable medium at least one compound Y, with at least one of said first and second compositions being in the form of a simple or multiple W/O/W emulsion comprising compound X or Y in its oily phase, and with at least one of said first and second composition additionally containing if necessary at least one catalyst or one peroxide.

Several layers of each of the first and second compositions can also be applied alternately on the keratinous substances.

More particularly, said method can comprise applying, on said keratinous substances, at least one composition in the form of a simple on multiple (W/O/W) emulsion containing, in a its oily phase, at least one compound X and at least one compound Y, said composition containing additionally if necessary at least one catalyst or one peroxide.

The composition applied can also be obtained by mixing, at the time of use, a first composition comprising at least compound X and a second composition comprising at least compound Y, at least one of the first and second compositions being in the form of a simple or multiple (W/O/W) emulsion, and at least one of the first and second compositions additionally comprising if necessary at least one catalyst or one peroxide.

As stated above, the compositions are advantageously in the form of a simple or W/O/W multiple emulsion.

According to one embodiment, at least one additional layer of at least one third composition comprising a cosmetically acceptable medium, and preferably at least one film-forming polymer and at least one organic solvent (or oily) medium or aqueous medium, is applied to the layer(s) of the composition(s) according to the invention comprising compounds X and Y and, if necessary, at least one catalyst or one peroxide, n order, for example, to improve the staying power and/or the comfort thereof.

According to a second aspect, the composition used in the process of the invention may be a single composition in the form of a simple or multiple (W/O/W) emulsion and containing compounds X and Y in the oily phase, with at least one of the compounds X and Y being in an encapsulated form, said composition additionally containing if necessary at least one catalyst or one peroxide.

Thus, the present invention also relates to a cosmetic composition in the form of a simple or multiple (W/O/W) emulsion, notably for care and/or make-up of keratinous substances containing in a physiologically acceptable medium at least one compound X in the oily phase, a compound Y in the oily phase as defined here with, said composition additionally containing if necessary at least one catalyst or one peroxide, and at least one of compounds X and Y being in an encapsulated form.

According to a preferred variant embodiment, the two compounds X and Y are present in separate encapsulated forms.

According to this embodiment, the two compounds X and Y can be packaged in one and the same composition while avoiding the risk of premature reaction between them. Said reaction only occurs at the moment when the composition is manipulated prior to or at the moment of its application on the keratinous substance. The encapsulated form or forms and compounds X and Y can react to form the expected film.

### Determination of the Matting Nature

The matting nature quantified by the value R is characterized by a measurement using a gonioreflectometer from the company Micromodule, equipped with an articulated arm from the company Newport ESP 300.

To do this, a mixture obtained from the composition(s) in accordance with the invention, in particular of first and second compositions according to the invention, in a 50/50 proportion, is spread onto a contrast card (Prufkarte type 24/5-250 cm² sold by the company Erichsen) using a mechanical film spreader (wet thickness of 150 microns).

The composition thus obtained is then dried overnight at a temperature of 37° C., and the reflection is then measured using a gonioreflectometer.

The result obtained is the ratio R between the specular reflection and the diffuse reflection. The value of R is proportionately smaller the greater the matting effect.

Advantageously, this film may be characterized by a matting parameter R with a value of less than 1.2, in particular less than 1, especially less than 0.8, or even less than 0.75.

Compounds X and Y

Silicone compound means a polyorganosiloxane compound, i.e. comprising at least two organosiloxane units, for example at least 5 organosiloxane units, notably at least 10 organosiloxane units. According to a particular embodiment, at least one of compounds X and Y, or compounds X and compounds Y are silicone compounds. Compounds X and Y can be aminated or non-aminated.

According to another embodiment, at least one of compounds X and Y is a polymer whose main chain is formed primarily of organosiloxane units. Among the silicone compounds mentioned below, some may display both film-forming and adhesive properties, depending for example on their proportion of silicone or depending on whether they are used mixed with a particular additive. It is therefore possible to adjust the film-forming properties or the adhesive properties of said compounds according to the proposed use, which is the case in particular for the so-called “room temperature vulcanization” reactive elastomeric silicones.

Compounds X and Y can react with each other at a temperature varying between room temperature and 180° C. Advantageously, compounds X and Y are capable of reacting together at room temperature (20±5° C.) and atmospheric pressure, or advantageously in the presence of a catalyst, by a hydrosilylation reaction or a condensation reaction, or a crosslinking reaction in the presence of a peroxide.

Polar Groups

According to a particular embodiment, at least one of compounds X and Y, for example compound X, bears at least one polar group that is able to form at least one hydrogen bond with keratinous substances.

By polar group, we mean a group having carbon atoms and hydrogen atoms in its chemical structure and at least one heteroatom (such as O, N, S and P), such that said group is able to establish at least one hydrogen bond with keratinous substances.

Compounds bearing at least one group that can form a hydrogen bond are particularly advantageous, as they endow the compositions containing them with better adherence on keratinous substances.

The polar group or groups borne by at least one of compounds X and Y is/are able to establish a hydrogen bond, and include either a hydrogen atom bound to an electronegative atom, or an electronegative atom for example an oxygen, nitrogen or sulphur atom. When the group has a hydrogen atom bound to an electronegative atom, the hydrogen atom can interact with another electronegative atom borne for example by another molecule, such as keratin, to form a hydrogen bond. When the group has an electronegative atom, the electronegative atom can interact with a hydrogen atom bound to an electronegative atom borne for example by another molecule, such as keratin, to form a hydrogen bond.

Advantageously, these polar groups can be selected from the following groups:
carboxylic acids —COOH,
alcohols, such as: —CH₂OH or —CH(R)OH, R being an alkyl radical having from 1 to 6 carbon atoms,
amino of formula —NR₁R₂, in which R₁ and R₂, which may be identical or different, represent an alkyl radical having from 1 to 6 carbon atoms or one of R₁ or R₂ denotes a hydrogen atom, and the other one of RI and R₂ represents an alkyl radical having from 1 to 6 carbon atoms,
pyridino,
amido of formula —NH—COR′ or —CO—NH—R′ in which R′ represents a hydrogen atom or an alkyl radical having from 1 to 6 carbon atoms,
pyrrolidino preferably selected from the groups of formula:

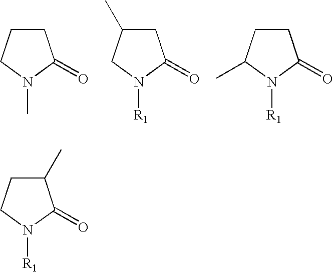

R₁ being an alkyl radical having from 1 to 6 carbon atoms,
carbamoyl of formula —O—CO—NH—R′ or —NH—CO—OR′, R′ being as defined above,
thiocarbamoyl such as —O—CS—NH—R′ or —NH—CS—OR′, R′ being as defined above,
ureyl such as —NR′—CO—N(R′)₂, the groups R′, which may be identical or different, being as defined above,
sulphonamido such as —NR′—S(═O)₂—R′, R′ corresponding to the above definition.

Preferably, these polar groups are present at a content less than or equal to 10 wt. % relative to the weight of each compound X or Y, preferably less than or equal to 5 wt. %, for example at a content ranging from 1 to 3 wt. %.

The polar group or groups can be located in the main chain of compound X and/or Y or can be pendant from the main chain or located at the ends of the main chain of compound X and/or Y.

1—Compounds X and Y Capable of Reacting by Hydrosilylation

According to one embodiment, the invention relates to a cosmetic kit for make-up or non therapeutic care of keratinous substance(s) comprising at least two compositions that are different and are packaged separately, the kit comprising at least one compound X, at least one compound Y, and at least one catalyst, with at least one of compounds X or Y being a silicone compound and said compounds X and Y being capable of reacting together by a hydrosilylation reaction in the presence of a catalyst, when they are brought into contact with one another, said kit being such that compounds X, Y and the catalyst are not present simultaneously in the same composition and that at least one of the compositions of said kit is in the form of a simple or multiple W/O/W emulsion in which said compound X or Y is present in the oily phase.

According to this embodiment, compounds X and Y are capable of reacting by hydrosilylation in the presence of a catalyst, said reaction being represented schematically in a simplified manner as follows:

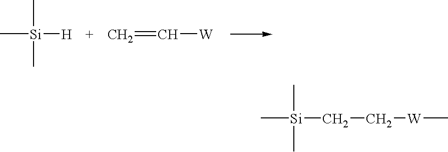

with W representing a carbon chain and/or silicone chain containing one or more unsaturated aliphatic groups.

In this case, compound X can be selected from silicone compounds comprising at least two unsaturated aliphatic groups. As an example, compound X can be a polyorganosiloxane comprising a silicone main chain whose unsaturated aliphatic groups are pendant from the main chain (side group) or located at the ends of the main chain of the compound (end group). These particular compounds will be called, hereinafter, polyorganosiloxanes with unsaturated aliphatic groups.

According to one embodiment, compound X and/or compound Y bear at least one polar group, as described above, capable of forming at least one hydrogen bond with keratinous substances. This polar group is advantageously carried by compound X, which has at least two unsaturated aliphatic groups.

According to one embodiment, compound X is selected from the polyorganosiloxanes comprising at least two unsaturated aliphatic groups, for example two or three vinyl or allyl groups, each attached to a silicon atom.

According to an advantageous embodiment, compound X is selected from the polyorganosiloxanes containing siloxane units of formula:

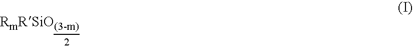

in which:
R represents a linear or cyclic, monovalent hydrocarbon group, having from 1 to 30 carbon atoms, preferably from 1 to 20, and better still from 1 to 10 carbon atoms, for example a short-chain alkyl radical, comprising for example from 1 to 10 carbon atoms, in particular a methyl radical or alternatively a phenyl group, preferably a methyl radical,
m is equal to 1 or 2 and
R′ represents:
   an unsaturated aliphatic hydrocarbon group having from 2 to 10, preferably from 3 to 5 carbon atoms, for example a vinyl group or a group —R″—CH═CHR′″ in which R″ is a divalent aliphatic hydrocarbon chain, having from 1 to 8 carbon atoms, bound to the silicon atom and R′″ is a hydrogen atom or an alkyl radical having from 1 to 4 carbon atoms, preferably a hydrogen atom; we may mention, as group R′, the vinyl and allyl groups and mixtures thereof; or
   an unsaturated cyclic hydrocarbon group having from 5 to 8 carbon atoms, for example a cyclohexenyl group.

Preferably R′ is an unsaturated aliphatic hydrocarbon group, preferably a vinyl group.

According to one embodiment, R represents an alkyl radical having from 1 to 10 carbon atoms or alternatively a phenyl group, and preferably a methyl radical, and R′ is a vinyl group.

According to a particular embodiment, the polyorganosiloxane also contains units of formula:

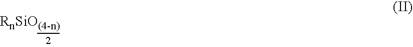

in which R is a group as defined previously, and n is equal to 1, 2 or 3.

According to a variant, compound X can be a silicone resin comprising at least two ethylenic unsaturations, said resin being capable of reacting with compound Y by hydrosilylation in the presence of a catalyst. We may mention for example the resins of type MQ or MT which themselves bear —CH═CH₂ unsaturated reactive end groups.

These resins are crosslinked organosiloxane polymers.

The class of the silicone resins is known by the name “MDTQ”, the resin being described in relation to the different siloxane monomer units that it contains, each of the letters “MDTQ” characterizing a type of unit.

The letter M represents the monofunctional unit of formula (CH₃)₃SiO_{1/2}, the silicon atom being bound to a single oxygen atom in the polymer comprising said unit.

The letter D denotes a bifunctional unit (CH₃)₂SiO_{2/2} in which the silicon atom is bound to two oxygen atoms.

The letter T represents a trifunctional unit of formula (CH₃)SiO_{3/2}.

In units M, D, T defined above, at least one of the methyl groups can be substituted with a group R other than the methyl group, such as a hydrocarbon radical (notably alkyl) having from 2 to 10 carbon atoms or a phenyl group or alternatively a hydroxyl group.

Finally, the letter Q denotes a tetrafunctional unit SiO_{4/2} in which the silicon atom is bound to four hydrogen atoms which are themselves attached to the rest of the polymer. As examples of said resins, we may mention the MT silicone resins such as poly(phenyl-vinylsilsesquioxane) such as that marketed under the reference SST-3PV1 by the company Gelest.

Preferably, compounds X have from 0.01 to 1 wt. % of unsaturated aliphatic groups.

Advantageously, compound X is selected from the polyorganopolysiloxanes, notably those comprising the siloxane units (I) and optionally (II) described previously.

Compound Y preferably has at least two free Si—H groups (hydrogenosilane groups).

Compound Y can be selected advantageously from the polyorganosiloxanes comprising at least one alkylhydrogenosiloxane unit of the following formula:

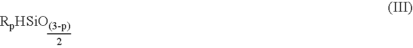

in which:

R represents a linear or cyclic, monovalent hydrocarbon group, having from 1 to 30 carbon atoms, for example an alkyl radical having from 1 to 30 carbon atoms, preferably from 1 to 20 and better still from 1 to 10 carbon atoms, in particular a methyl radical, or alternatively a phenyl group and p is equal to 1 or 2. Preferably R is a hydrocarbon group, preferably methyl.

These polyorganosiloxane compounds Y with alkylhydrogenosiloxane units can additionally contain units of formula:

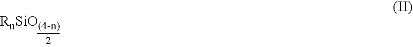

as defined above.

Compound Y can be a silicone resin comprising at least one unit selected from the units M, D, T, Q as defined above and comprising at least one Si—H group such as the poly(methyl-hydridosilsesquioxane) marketed under the reference SST-3MH1.1 by the company Gelest.

Preferably, these polyorganosiloxane compounds Y have from 0.5 to 2.5 wt. % of Si—H groups.

Advantageously, the radicals R represent a methyl group in formulae (I), (II), (III) above.

Preferably, these polyorganosiloxanes Y have end groups of formula (CH₃)₃SiO_{1/2}.

Advantageously, the polyorganosiloxanes Y have at least two alkylhydrogenosiloxane units of formula —(H₃C)(H)SiO— and optionally include —(H₃C)₂SiO— units.

These polyorganosiloxane compounds Y with hydrogenosilane groups are described for example in document EP 0465744.

According to one variant, compound X is selected from the organic oligomers or polymers (by organic, we mean compounds whose main chain is not a silicone chain, preferably compounds not containing silicon atoms) or from hybrid organic/silicone polymers or oligomers, said oligomers or polymers bearing at least 2 unsaturated reactive aliphatic groups, compound Y being selected from the polyorganosiloxanes Y with hydrogenosilane groups mentioned above.

According to one embodiment, the organic or hybrid organic/silicone compounds X bearing at least 2 unsaturated reactive aliphatic groups, have at least one polar group as described above.

Compound X, of organic nature, can then be selected from the vinylic, (meth)acrylic polymers or oligomers, polyesters, polyurethanes and/or polyureas, polyethers, perfluoropolyethers, polyolefins such as polybutene, polyisobutylene, dendrimers or organic hyperbranched polymers, or mixtures thereof.

In particular, the organic polymer or the organic moiety of the hybrid polymer can be selected from the following polymers:

a) polyesters with ethylenic unsaturation(s):

This is a group of polymers of the polyester type having at least 2 ethylenic double bonds, randomly distributed in the main chain of the polymer. These unsaturated polyesters are obtained by polycondensation of a mixture:
of linear or branched aliphatic or cycloaliphatic dicarboxylic acids notably having 3 to 50 carbon atoms, preferably from 3 to 20 and better still from 3 to 10 carbon atoms, such as adipic acid or sebacic acid, of aromatic dicarboxylic acids notably having from 8 to 50 carbon atoms, preferably from 8 to 20 and better still from 8 to 14 carbon atoms, such as phthalic acids, notably terephthalic acid, and/or of dicarboxylic acids derived from dimers of fatty acids with ethylenic unsaturations such as the dimers of oleic or linoleic acids described in application EP-A-959 066 (paragraph [0021]) marketed under the designations Pripol® by the company Unichema or Empol® by the company Henkel, all said diacids having to be free from polymerizable ethylenic double bonds,
of linear or branched aliphatic or cycloaliphatic diols notably having from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols having from 6 to 50 carbon atoms, preferably from 6 to 20 and better still from 6 to 15 carbon atoms such as bisphenol A and bisphenol B, and/or of diol dimers resulting from reduction of dimers of fatty acids as defined previously, and
of one or more dicarboxylic acids or their anhydrides having at least one polymerizable ethylenic double bond and having from 3 to 50 carbon atoms, preferably from 3 to 20 and better still from 3 to 10 carbon atoms, such as maleic acid, fumaric acid or itaconic acid.

b) polyesters with (meth)acrylate side and/or end groups:

This is a group of polymers of the polyester type obtained by polycondensation of a mixture:
of linear or branched aliphatic or cycloaliphatic dicarboxylic acids notably having from 3 to 50 carbon atoms, preferably from 3 to 20 and better still from 3 to 10 carbon atoms, such as adipic acid or sebacic acid, of aromatic dicarboxylic acids notably having from 8 to 50 carbon atoms, preferably from 8 to 20 and better still from 8 to 14 carbon atoms, such as phthalic acids, notably terephthalic acid, and/or of dicarboxylic acids derived from dimers of fatty acids with an ethylenic unsaturation such as the dimers of oleic or linoleic acids described in application EP-A-959 066 (paragraph [0021]) marketed under the designations Pripol® by the company Unichema or Empol® by the company Henkel, all said diacids having to be free from polymerizable ethylenic double bonds,
of linear or branched aliphatic or cycloaliphatic diols notably having from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols having from 6 to 50 carbon atoms, preferably from 6 to 20 and better still from 6 to 15 carbon atoms such as bisphenol A and bisphenol B, and
of at least one ester of (meth)acrylic acid and of a diol or polyol having from 2 to 20 carbon atoms, preferably from 2 to 6 carbon atoms, such as 2-hydroxyethyl (meth)acrylate, 2-hydroxypropyl (meth)acrylate and glycerol methacrylate.

These polyesters differ from those described above in section a) by the fact that the ethylenic double bonds are not located in the main chain but on side groups or at the end of the chains. These ethylenic double bonds are those of the (meth)acrylate groups present in the polymer.

Such polyesters are marketed for example by the company UCB under the designations EBECRYL® (EBECRYL® 450: molecular weight 1600, on average 6 acrylate functions per molecule, EBECRYL® 652: molecular weight 1500, on average 6 acrylate functions per molecule, EBECRYL® 800: molecular weight 780, on average 4 acrylate functions per molecule, EBECRYL® 810: molecular weight 1000, on average 4 acrylate functions per molecule, EBECRYL® 50 000: molecular weight 1500, on average 6 acrylate functions per molecule).

c) polyurethanes and/or polyureas with (meth)acrylate groups, obtained by polycondensation:
of aliphatic, cycloaliphatic and/or aromatic diisocyanates, triisocyanates and/or polyisocyanates notably having from 4 to 50, preferably from 4 to 30 carbon atoms, such as hexamethylenediisocyanate, isophoronediisocyanate, toluenediisocyanate, diphenylmethanediisocyanate or isocyanurates of formula:

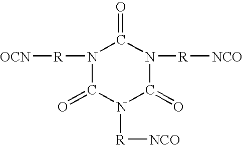

resulting from the trimerization of 3 molecules of diisocyanates OCN—R—CNO, where R is a linear, branched or cyclic hydrocarbon radical having from 2 to 30 carbon atoms;
of polyols, notably of diols, free from polymerizable ethylenic unsaturations, such as 1,4-butanediol, ethylene glycol or trimethylolprdpane, and/or of polyamines, notably of aliphatic, cycloaliphatic and/or aromatic diamines, notably having from 3 to 50 carbon atoms, such as ethylenediamine or hexamethylenediamine, and
of at least one ester of (meth)acrylic acid and of a diol or polyol having from 2 to 20 carbon atoms, preferably from 2 to 6 carbon atoms, such as 2-hydroxyethyl (meth)acrylate, 2-hydroxypropyl (meth)acrylate and glycerol methacrylate.

These polyurethanes/polyureas with acrylate groups are marketed for example under the designation SR 368 (tris(2-hydroxyethyl)isocyanurate-triacrylate) or CRAYNOR® 435 by the company CRAY VALLEY, or under the designation EBECRYL® by the company UCB (EBECRYL® 210: molecular weight 1500, 2 acrylate functions per molecule, EBECRYL® 230: molecular weight 5000, 2 acrylate functions per molecule, EBECRYL® 270: molecular weight 1500, 2 acrylate functions per molecule, EBECRYL® 8402: molecular weight 1000, 2 acrylate functions per molecule, EBECRYL® 8804: molecular weight 1300, 2 acrylate functions per molecule, EBECRYL® 220: molecular weight 1000, 6 acrylate functions per molecule, EBECRYL® 2220: molecular weight 1200, 6 acrylate functions per molecule, EBECRYL® 1290: molecular weight 1000, 6 acrylate functions per molecule, EBECRYL® 800: molecular weight 800, 6 acrylate functions per molecule).

We may also mention the water-soluble aliphatic diacrylate polyurethanes marketed under the designations EBECRYL® 2000, EBECRYL® 2001 and EBECRYL® 2002, and the diacrylate polyurethanes in aqueous dispersion marketed under the trade names IRR® 390, IRR® 400, IRR® 422 IRR® 424 by the company UCB.

d) polyethers with (meth)acrylate groups obtained by esterification, by (meth)acrylic acid, of the hydroxyl end groups of homopolymers or of C₁₋₄ alkylene glycol copolymers, such as polyethylene glycol, polypropylene glycol, copolymers of ethylene oxide and of propylene oxide preferably having a weight-average molecular weight below 10 000, polyethoxylated or polypropoxylated trimethylolpropane.

Di(meth)acrylate polyoxyethylenes of suitable molecular weight are marketed for example under the designations SR 259, SR 344, SR 610, SR 210, SR 603 and SR 252 by the company CRAY VALLEY or under the designation EBECRYL® 11 by UCB. Polyethoxylated trimethylolpropane triacrylates are marketed for example under the designations SR 454, SR 498, SR 502, SR 9035, SR 415 by the company CRAY VALLEY or under the designation EBECRYL® 160 by the company UCB. Polypropoxylated trimethylolpropane triacrylates are marketed for example under the designations SR 492 and SR 501 by the company CRAY VALLEY.

e) epoxyacrylates obtained by reaction between
at least one diepoxide selected for example from:
   (i) bisphenol A diglycidyl ether,
   (ii) a diepoxy resin resulting from the reaction between bisphenol A diglycidyl ether and epichlorohydrin,
   (iii) an epoxyester resin with α,ω-diepoxy end groups resulting from the condensation of a dicarboxylic acid having from 3 to 50 carbon atoms with a stoichiometric excess of (i) and/or (ii),
   (iv) an epoxyether resin with α,ω-diepoxy end groups resulting from the condensation of a diol having from 3 to 50 carbon atoms with a stoichiometric excess of (i) and/or (ii),
   (v) natural or synthetic oils bearing at least 2 epoxide groups, such as epoxidized soya oil, epoxidized linseed oil and epoxidized vernonia oil,
   (vi) a phenol-formaldehyde polycondensate (Novolac® resin), of which the end groups and/or side groups have been epoxidized, and
one or more carboxylic acids or carboxylic polyacids having at least one ethylenic double bond at α,β of the carboxyl group such as (meth)acrylic acid or crotonic acid or esters of (meth)acrylic acid and of a diol or polyol having from 2 to 20 carbon atoms, preferably from 2 to 6 carbon atoms such as 2-hydroxyethyl(meth)acrylate.

Such polymers are marketed for example under the designations SR 349, SR 601, CD 541, SR 602, SR 9036, SR 348, CD 540, SR 480, CD 9038 by the company CRAY VALLEY, under the designations EBECRYL® 600 and EBECRYL® 609, EBECRYL® 150, EBECRYL® 860, EBECRYL® 3702 by the company UCB and under the designations PHOTOMER® 3005 and PHOTOMER® 3082 by the company HENKEL.

f) (C₁₋₅₀ alkyl) poly(meth)acrylates, said alkyl being linear, branched or cyclic, bearing at least two functions with ethylenic double bond carried by the lateral and/or terminal hydrocarbon chains.

Such copolymers are marketed for example under the designations IRR® 375, OTA® 480 and EBECRYL® 2047 by the company UCB.

g) polyolefins such as polybutene, polyisobutylene,

h) perfluoropolyethers with acrylate groups obtained by esterification, for example by (meth)acrylic acid, of perfluoropolyethers bearing hydroxyl side and/or end groups.

These α,ω-diol perfluoropolyethers are described notably in EP-A-1057849 and are marketed by the company AUSIMONT under the designation FOMBLIN® Z DIOL.

i) dendrimers and hyperbranched polymers bearing (meth)acrylate or (meth)acrylamide end groups obtained respectively by esterification or amidation of dendrimers and of hyperbranched polymers with hydroxyl or amino terminal functions, by (meth)acrylic acid.

The dendrimers (from the Greek dendron=tree) are “tree-like” polymer molecules, i.e. highly branched, invented by D.A. Tomalia and his team at the beginning of the 1990's (Donald A. Tomalia et al., Angewandte Chemie, Int. Engl. Ed., Vol. 29, No. 2, pages 138-175). They are structures constructed around a, generally polyvalent, central unit. Branched chain-extending units are arranged according to a perfectly defined structure around this central unit, thus giving rise to symmetrical, monodispersed macromolecules having a well-defined chemical and stereochemical structure. Dendrimers of the polyamidoamine type are marketed for example under the name STARBURST® by the company DENDRITECH.

The hyperbranched polymers are polycondensates, generally of the polyester, polyamide or polyethyleneamine type, obtained from multifunctional monomers, which have a tree-like structure similar to that of the dendrimers but far less regular than the latter (see for example WO-A-93/17060 and WO 96/12754).

The company PERSTORP markets hyperbranched polyesters under the name BOLTORN®. Hyperbranched polyethyleneamines are available under the name COMBURST® from the company DENDRITECH. Hyperbranched poly(esteramide)s with hydroxyl end groups are marketed by the company DSM under the name HYBRANE®.

These dendrimers and hyperbranched polymers, esterified or amidated by acrylic and/or methacrylic acid, differ from the polymers described in sections a) to h) above by the very large number of ethylenic double bonds present. This increased functionality, generally greater than 5, makes them particularly useful in enabling them to act as a “crosslinking node”, i.e. a multiple crosslinking site.

It is therefore possible to use these dendritic and hyperbranched polymers in association with one or more of the above polymers and/or oligomers a) to h).

1a—Additional Reactive Compounds

According to one embodiment, the compositions containing compound X and/or Y can additionally comprise an additional reactive compound such as:
organic or mineral particles having on their surface at least 2 unsaturated aliphatic groups—we may mention for example the silicas surface-treated for example with silicone compounds with vinylic groups such as for example cyclotetramethyltetravinylsiloxane-treated silica,
silazane compounds such as hexamethyldisilazane.

1b—Catalyst

The hydrosilylation reaction takes place in the presence of a catalyst which can be present with one or other of the compounds X or Y or can be present on its own. For example, this catalyst can be present in the composition in an encapsulated form if the two compounds X and Y, which it must cause to interact, are present in this same composition in an unencapsulated form or conversely it can be contained there in an unencapsulated form if at least one of compounds X and Y is present in the composition in an encapsulated form. The catalyst is preferably based on platinum or tin.

We may mention for example platinum-based catalysts deposited on a support of silica gel or of powdered charcoal, platinum chloride, salts of platinum and of chloroplatinic acids.

The chloroplatinic acids are preferably used in hexahydrate or anhydrous form, which are easily dispersible in organosilicone media.

We may also mention platinum complexes, such as those based on chloroplatinic acid hexahydrate and divinyl tetramethyldisiloxane.

The catalyst can be present at a content in the range from 0.0001 to 20 wt. % relative to the total weight of the composition containing it.

Compounds X and/or Y can be combined with polymerization inhibitors or retarders, and more particularly inhibitors of the catalyst. Non-limitatively, we may mention cyclic polymethylvinylsiloxanes, and in particular tetravinyl tetramethyl cyclotetrasiloxane, acetylenic alcohols, preferably volatile, such as methylisobutynol.

The presence of ionic salts, such as sodium acetate, can have an influence on the rate of polymerization of the compounds.

As an example of a combination of compounds X and Y reacting by hydrosilylation in the presence of a catalyst, we may mention the following references offered by the company Dow Corning: DC 7-9800 Soft Skin Adhesive Parts A & B, as well as the combination of the following mixtures A and B prepared by Dow Corning:

Mixture A:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| 1,3-Diethenyl-1,1,3,3- | 68478-92-2 | Trace | Catalyst |
| Tetramethyldisiloxane | | | |
| complexes | | | |
| Tetramethyldivinyldisiloxane | 2627-95-4 | 0.1-1 | Polymer |

### Mixture B:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| Dimethyl, | 68037-59-2 | 1-10 | Polymer |
| Methylhydrogen | | | |
| Siloxane, trimethylsiloxy- | | | |
| terminal | | | |

Advantageously, compounds X and Y are selected from silicone compounds capable of reacting by hydrosilylation in the presence of a catalyst; in particular compound X is selected from the polyorganosiloxanes comprising units of formula (I) described above and compound Y is selected from organosiloxanes comprising alkylhydrogenosiloxane units of formula (III) described above.

According to a particular embodiment, compound X is a polydimethylsiloxane with vinylic end groups, and compound Y is a polymethylhydrogenosiloxane.

2/Compounds X and Y Capable of Reacting by condensation

According to one embodiment, the invention relates to a cosmetic kit for make-up or non therapeutic care of keratinous substance(s) comprising at least two compositions that are different and are packaged separately, the kit comprising at least one compound X, at least one compound Y, and optionally at least one catalyst, with at least one of compounds X or Y being a silicone compound and said compounds X and Y being capable of reacting together by a condensation reaction, if necessary in the presence of a catalyst, when they are brought into contact with one another, said kit being such that compounds X and Y and the catalyst, when it is present, are not present simultaneously in the same composition and that at least one of the compositions of said kit is in the form of a simple or multiple W/O/W emulsion in which said compound X or Y is present in the oily phase.

According to this embodiment, compounds X and Y are capable of reacting by condensation, either in the presence of water (hydrolysis) by reaction of 2 compounds bearing alkoxysilane groups, or by so-called “direct” condensation by reaction of a compound bearing alkoxysilane group(s) and a compound bearing silanol group(s) or by reaction of 2 compounds bearing silanol group(s).

When the condensation is carried out in the presence of water, the latter can in particular be the ambient humidity, the residual water of the skin, of the lips, of the eyelashes and/or of the nails, or water from an external source, for example by prior moistening of the keratinous substance (for example by an atomizer, by natural or artificial tears).

In this manner of reaction by condensation, compounds X and Y, which may be identical or different, can therefore be selected from silicone compounds whose main chain contains at least two alkoxysilane groups and/or at least two silanol (Si—OH) side groups or end groups.

According to one embodiment, compound X and/or compound Y bears at least one polar group, as described above, capable of forming at least one hydrogen bond with keratinous substances.

According to an advantageous embodiment, compounds X and/or Y are selected from the polyorganosiloxanes comprising at least two alkoxysilane groups. By “alkoxysilane group”, we mean a group comprising at least one —Si—OR moiety, R being an alkyl group having from 1 to 6 carbon atoms.

Compounds X and Y are notably selected from the polyorganosiloxanes comprising alkoxysilane end groups, more specifically those which have at least 2 alkoxysilane end groups, preferably trialkoxysilane end groups.

These compounds X and/or Y preferably mostly comprise units of formula:

R⁹ₛSiO_{(4-s/2}, (IV)

in which the groups R⁹ represent, independently of one another, a radical selected from alkyl groups having from 1 to 6 carbon atoms, phenyl groups, fluoroalkyl groups, and s is equal to 0, 1, 2 or 3. Preferably, groups R⁹ represent, independently of one another, an alkyl group having from 1 to 6 carbon atoms. As alkyl group, we may notably mention methyl, propyl, butyl, hexyl and mixtures thereof, preferably methyl or ethyl. As fluoroalkyl group, we may mention 3,3,3-trifluoropropyl.

According to a particular embodiment, compounds X and Y, which may be identical or different, are polyorganosiloxanes comprising units of formula:

(R⁹₂SiO₂)_{f}— (V)

in which R⁹ is as described above, preferably R⁹ is a methyl radical, and f is such that the polymer advantageously has a viscosity at 25° C. in the range from 0.5 to 3000 Pa·s, preferably in the range from 5 to 150 Pa·s; for example f can range from 2 to 5000, preferably from 3 to 3000, and more preferably from 5 to 1000.

These polyorganosiloxane compounds X and Y contain at least 2 trialkoxysilane end groups per molecule of polymer, said groups having the following formula

-ZiR¹ₓ(OR)₃₋ₓ, (VI)

in which:

the radicals R represent, independently, a methyl, ethyl, n-propyl, isopropyl, n-butyl, sec-butyl, isobutyl group, preferably a methyl or ethyl group,

R¹ is a methyl or ethyl group,

x is equal to 0 or 1, preferably x is equal to 0 and

Z is selected from: the divalent hydrocarbon groups that do not have an ethylenic unsaturation and have from 1 to 18 carbon atoms, preferably from 2 to 18 carbon atoms (alkylene groups), the combinations of divalent hydrocarbon radicals and siloxane segments of the following formula (IX):

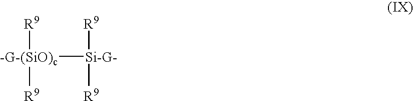

R⁹ being as described above, G is a divalent hydrocarbon radical without an ethylenic unsaturation and having from 1 to 18 carbon atoms, preferably from 2 to 18 carbon atoms and c is an integer in the range from 1 to 6.

Z and G can notably be selected from the alkylene groups such as methylene, ethylene, propylene, butylene, pentylene, hexylene, the arylene groups such as phenylene.

Preferably, Z is an alkylene group, and more preferably ethylene.

These polymers can have on average at least 1.2 trialkoxysilane end groups or terminal chains per molecule, and preferably on average at least 1.5 trialkoxysilane end groups per molecule. These polymers that can have at least 1.2 trialkoxysilane end groups per molecule, some can include other types of end groups such as end groups of formula CH₂═CH—SiR⁹₂— or of formula R⁶₃—Si—, in which R⁹ is as defined previously and each group R⁶ is selected independently from the R⁹ or vinyl groups. As examples of said end groups, we may mention the trimethoxysilane, triethoxysilane, vinyldimethoxysilane and vinylmethyloxyphenylsilane groups.

Such polymers are notably described in documents U.S. Pat. No. 3,175,993, U.S. Pat. No. 4,772,675, U.S. Pat. No. 4,871,827, U.S. Pat. No. 4,888,380, U.S. Pat. No. 4,898,910, U.S. Pat. No. 4,906,719 and U.S. Pat. No. 4,962,174, the contents of which are incorporated by reference in the present application.

We may mention, as compound X and/or Y, in particular the polyorganosiloxanes selected from the polymers of formula:

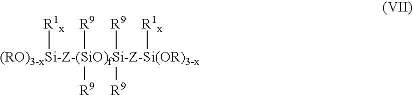

in which R, R¹, R⁹, Z, x and f are as described above.

Compounds X and/or Y can also include a mixture of polymers of formula (VII) above with polymers of the following formula (VIII):

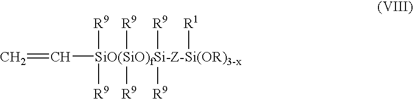

in which R, R¹, R⁹, Z, x, and f are as described above.

When the polyorganosiloxane compound X and/or Y with alkoxysilane group(s) includes said mixture, the various polyorganosiloxanes are present at contents such that the organosilyl terminal chains represent less than 40%, preferably less than 25% in number of terminal chains.

Polyorganosiloxane compounds X and/or Y that are particularly preferred are those of formula (VII) described above. Such compounds X and/or Y are described for example in document WO 01/96450.

As stated above, compounds X and Y can be identical or different.

In particular, compounds X and Y can represent a mixture of polydimethylsiloxanes with methoxysilane groups.

According to a variant, one of the 2 reacting compounds X or Y is of silicone character and the other is of organic character. For example, compound X is selected from organic oligomers or polymers or hybrid organic/silicone oligomers or polymers, said polymers or oligomers comprising at least two alkoxysilane groups, and Y is selected from silicone compounds such as the polyorganosiloxanes described above. In particular, the organic oligomers or polymers are selected from the vinylic, (meth)acrylic oligomers or polymers, polyesters, polyamides, polyurethanes and/or polyureas, polyethers, polyolefins, perfluoropolyethers, dendrimers and hyperbranched organic polymers, and mixtures thereof.

According to one embodiment, compound X of organic character or of hybrid organic/silicone character bears at least one polar group, as described above, capable of forming at least one hydrogen bond with the keratinous substance.

The organic polymers of vinylic or (meth)acrylic character, bearing alkoxysilane side groups, can in particular be obtained by copolymerization of at least one vinylic or (meth)acrylic organic monomer with a (meth)acryloxypropyltrimethoxysilane, a vinyltrimethoxysilane, a vinyltriethoxysilane, an allyltrimethoxysilane etc.

We may mention for example the (meth)acrylic polymers described in the document of KUSABE, M, Pitture e Verniei—European Coating; 12-B, pages 43-49, 2005, and notably the polyacrylates with alkoxysilane groups with the designation MAX from Kaneka or those described in the work by PROBSTER, M, Adhesion-Kleben & Dichten, 2004, 481 (1-2), pages 12-14.

The organic polymers resulting from a polycondensation or a polyaddition, such as polyesters, polyamides, polyurethanes and/or polyureas, polyethers, and bearing alkoxysilane side and/or end groups, can result for example from reaction of an oligomeric prepolymer as described above with one of the following silane reaction partners bearing at least one alkoxysilane group: aminopropyltrimethoxysilane, aminopropyltriethoxysilane, aminoethyl aminopropyl trimethoxysilane, glycidoxypropyltrimethoxysilane, glycidoxypropyltriethoxysilane, epoxycyclohexylethyltrimethoxysilane, mercaptopropyltrimethoxysilane.

Examples of polyethers and polyisobutylenes with alkoxysilane groups are described in the work by KUSABE, M., Pitture e Verniei—European Coating; 12-B, pages 43-49, 2005. As examples of polyurethanes with alkoxysilane end groups, we may mention those described in the document PROBSTER, M., Adhesion-Kleben & Dichten, 2004, 481 (1-2), pages 12-14 or alternatively those described in the document LANDON, S., Pitture e Verniei Vol. 73, No. 11, pages 18-24, 1997 or in the document HUANG, Mowo, Pitture e Verniei Vol. 5, 2000, pages 61-67, and we may notably mention the polyurethanes with alkoxysilane groups from OSI-WITCO-GE.

As polyorganosiloxane compounds X and/or Y, we may mention the resins of type MQ or MT which themselves bear alkoxysilane and/or silanol end groups, for example the poly(isobutylsilsesquioxane) resins functionalized with silanol groups offered under reference SST-S7C41 (three Si—OH groups) by the company Gelest.

2a—Additional Reactive Compound

According to one embodiment, compound X and/or Y can additionally be combined with an additional reactive compound comprising at least two alkoxysilane or silanol groups.

We may mention for example:
one or more organic or mineral particles with alkoxysilane and/or silanol groups on their surface, for example fillers surface-treated with said groups.

2b—Catalyst

The condensation reaction can take place in the presence of a metal-based catalyst which can be present with one or other of the compounds X or Y or can be present on its own. For example, said catalyst can be present in the composition in an encapsulated form if the two compounds X and Y, which it is to cause to interact, are present in this same composition in an unencapsulated form or conversely it can be present there in an unencapsulated form if at least one of compounds X and Y is present in the composition in an encapsulated form. The catalyst for use in this type of reaction is preferably a titanium-based catalyst.

We may notably mention the catalysts based on tetraalkoxytitanium of formula:

Ti(OR²)_{y}(OR³)_{4-y},

in which R² is selected from the tertiary alkyl radicals such as tert-butyl, tert-amyl and 2,4-dimethyl-3-pentyl; R³ represents an alkyl radical having from 1 to 6 carbon atoms, preferably a methyl, ethyl, n-propyl, isopropyl, n-butyl, sec-butyl, hexyl group and y is a number in the range from 3 to 4, preferably from 3.4 to 4.

The catalyst can be present at a content ranging from 0.0001 to 20 wt. % relative to the total weight of the composition containing it.

2c—Diluent

The compositions that can be used, comprising X and/or Y, can additionally include a volatile silicone oil (or diluent) for lowering the viscosity of the composition. Said oil can be selected from the short-chain linear silicones such as hexamethyldisiloxane, octamethyltrisiloxane, cyclic silicones such as octamethylcyclotetrasiloxane, decamethylcyclopentasiloxane and mixtures thereof.

This silicone oil can represent from 5 to 95 wt. %, preferably from 10 to 80 wt. % relative to the weight of each composition.

As an example of a combination of compounds X and Y bearing alkoxysilane groups and reacting by condensation, we may mention the combination of the following mixtures A′ and B′ produced by the company Dow Corning:

Mixture A′:

| Ingredient (INCI name) | CAS No. | Contents (%) | Function |
|---|---|---|---|
| Bis- | PMN87176 | 25-45 | Polymer |
| Trimethoxysiloxyethyl | | | |
| Tetramethyldisiloxyethyl | | | |
| Dimethicone (1) | | | |
| Silica Silylate | 68909-20-6 | 5-20 | Filler |
| Disiloxane | 107-46-0 | 30-70 | Solvent |

Mixture B′:

| Ingredient (INCI name) | CAS No. | Contents (%) | Function |
|---|---|---|---|
| Disiloxane | 107-46-0 | 80-99 | Solvent |
| Tetra T Butyl Titanate | — | 1-20 | Catalyst |

It should be noted that compounds X and Y, identical, are combined in mixture A′ (cf. (1)).

3/Crosslinking in the Presence of Peroxide:

According to one embodiment, the invention relates to a cosmetic kit for make-up or non therapeutic care of keratinous substance(s) comprising at least two compositions that are different and are packaged separately, the kit comprising at least one compound X, at least one compound Y, and at least one peroxide, with at least one of compounds X or Y being a silicone compound and said compounds X and Y being capable of reacting together by a crosslinking reaction in the presence of a peroxide, when they are brought into contact with one another, said kit being such that compounds X, Y and the peroxide are not present simultaneously in the same composition and that at least one of the compositions of said kit is in the form of a simple or multiple W/O[W emulsion in which said compound X or Y is present in the oily phase.

This reaction is preferably effected by heating to a temperature greater than or equal to 50° C., preferably greater than or equal to 80° C., and up to 120° C.

Compounds X and Y, which may be identical or different, have in this case at least two —CH₃ side groups and/or at least two side chains bearing a —CH₃ group.

Compounds X and Y are preferably silicone compounds and can be selected for example from the non-volatile linear polydimethylsiloxanes of high molecular weight, having a degree of polymerization above 6 and with at least two —CH₃ side groups attached to the silicon atom and/or at least two side chains bearing a —CH₃ group. We may mention for example the polymers described in the Catalogue “Reactive Silicones” of the company Gelest Inc., Edition 2004, page 6, and notably the copolymers (also called gums) of vinylmethylsiloxane-dimethylsiloxane of molecular weight in the range from 500 000 to 900 000 and notably with viscosity above 2 000 000 cSt.

As peroxides that can be used in the invention, we may mention benzoyl peroxide, 2,4-dichlorobenzoyl peroxide and mixtures thereof.

According to one embodiment, the hydrosilylation reaction in the presence of a catalyst, or the condensation reaction, or alternatively the crosslinking reaction in the presence of a peroxide, between compounds X and Y is accelerated by supply of heat, for example by raising the temperature of the system between 25° C. and 180° C.

In general, regardless of the type of reaction by which compounds X and Y react with one another, the molar percentage of X relative to the total of compounds X and Y, i.e. the ratio X/(X+Y)×100, can vary from 5 to 95%, preferably from 10 to 90%, and more preferably from 20 to 80%.

Similarly, the molar percentage of Y relative to the total of compounds X and Y, i.e. the ratio Y/(X+Y)×100, can vary from 5 to 95%, preferably from 10 to 90%, and more preferably from 20 to 80%.

Compound X can have a weight-average molecular weight (Mw) in the range from 150 to 1 000 000, preferably from 200 to 800 000, more preferably from 200 to 250 000.

Compound Y can have a weight-average molecular weight (Mw) in the range from 200 to 1 000 000, preferably from 300 to 800 000, more preferably from 500 to 250 000.

Compound X can represent from 0.1 to 95 wt. % relative to the total weight of the composition containing it, preferably from 1 to 90%, and more preferably from 5 to 80%.

Compound Y can represent from 0.1 to 95 wt. % relative to the total weight of the composition containing it, preferably from 1 to 90%, and more preferably from 5 to 80%.

The ratio of compound X to compound Y can be varied so as to adjust the reaction rate and therefore the rate of formation of the film or alternatively so as to adapt the properties of the film formed (for example its adhesive properties) according to the intended application.

In particular, compounds X and Y can be present at a molar ratio X/Y in the range from 0.05 to 20 and preferably from 0.1 to 10.

Compounds X and Y can advantageously be combined with at least one filler. Thus, the kit according to the invention can for example include, in at least one of the compositions, a filler selected from silica or surface-treated silica.

As pointed out previously, according to one embodiment of the invention, compounds X and Y can be used in the form of a single composition which then contains at least one of them or, if applicable, the catalyst or the peroxide if necessary for their interaction, in an encapsulated form.

Within the scope of the present invention, consideration is given more particularly to the encapsulated forms of the core/shell type, also called microcapsules or nanocapsules, in which the shell is of polymeric character and the core contains compound X, compound Y, one of its compounds X and Y possibly being encapsulated with the catalyst or the peroxide if necessary for the interaction of the two compounds. In the case when this catalyst is not encapsulated with one or other of the compounds X or Y, it is present in the cosmetic composition containing the encapsulated forms.

Numerous techniques are currently available for making microcapsules or nanocapsules of this type.

However, according to a preferred embodiment, the encapsulated forms considered according to the invention are nanocapsules and are obtained by a technique called solvent nanoprecipitation, notably described in documents EP 274 961 and EP 1 552 820.

More particularly, the shell of the nanocapsules of compound X or Y, employed according to the invention, is of polymeric character, not crosslinked, not water-soluble and not soluble in the capsule core.

In general, all the polymers, of natural or synthetic origin, soluble in a solvent that is not miscible with water, and notably those having a melting point below the boiling point of water at atmospheric pressure (100° C.), may be suitable.

These polymers can be biodegradable, for example polyesters, or non-biodegradable.

By way of illustration of polymers that are suitable for the invention, we may notably mention:
C₂-C₁₂ alkyl cyanoacrylate polymers
polymers formed by poly-L-lactides, poly-DL-lactides, polyglycolides and the corresponding copolymers,
polycaprolactones,
polymers of 3-hydroxybutyric acid,
copolymers of vinyl chloride and vinyl acetate,
copolymers of methacrylic acid and methacrylic ester, notably of methacrylic acid and of methacrylate,
polyvinyl acetophthalate,
cellulose acetophthalate,
polyvinylpyrrolidone-vinyl acetate copolymer,
polyethylenevinyl acetates,
polyacrylonitriles,
polyacrylamides,
polyethylene glycols,
poly-(C₁ to C₄ hydroxyalkyl methacrylate)
esters of cellulose and C₁-C₄ carboxylic acid,
polystyrene and copolymers of styrene and maleic anhydride, copolymers of styrene and acrylic acid, styrene ethylene/butylene-styrene block terpolymers, styrene-ethylene/propylene-styrene block terpolymers,
styrene alkyl-alcohol oligomers,
terpolymers of ethylene, vinyl acetate and maleic anhydride,
polyamides,
polyethylenes,
polypropylenes,
organopolysiloxanes including polydimethylsiloxanes,
poly(alkylene adipate),
polyol polyesters,
polysilsesquioxane silicone polymers,
dendritic polyesters with a hydroxyl terminal function,
polymers that are water-dispersible but are nevertheless soluble in solvents that are not miscible with water, for example: polyesters, poly(ester amides), polyurethanes and vinyl copolymers bearing carboxylic and/or sulphonic acid functions and in particular those described in document FR 2 787 729,
block copolymers insoluble in water at room temperature and solid at room temperature, having at least one block of one of the aforementioned polymers, and
mixtures thereof.

These polymers or copolymers can have a weight-average molecular weight between 1000 and 500 000 and in particular between 1500 and 100 000.

The following are quite particularly suitable for the invention: poly(alkylene adipate), organopolysiloxanes, polycaprolactones, cellulose acetophthalate, cellulose acetobutyrate, cellulose esters, polystyrene and its derivatives, and especially polycaprolactones.

Of course, a person skilled in the art is able, on the basis of his knowledge, to adjust the molecular weight of the polymer selected with respect to its concentration in the solvent so as have a mixture viscosity compatible with satisfactory emulsification.

With regard to the lipophilic core, it can contain at least one oil, in addition to compound X or compound Y. Said oil can be selected from the oils described hereunder for the oily phase. The oil is preferably a silicone oil.

According to a variant of the invention, the encapsulated forms of compound X or compound Y can be coated with a lamellar phase.

Regarding the operating procedure for production of nanocapsules suitable for the invention, a person skilled in the art can notably refer to the teaching in document EP 1 552 820 cited previously. The choice of the necessary surfactants as well as the carrying out of the method requires the knowledge of a person skilled in the art.

Physiologically Acceptable Medium

As stated above, the compositions according to the invention include a physiologically acceptable medium, i.e. a non-toxic medium that can be applied on keratinous substances of human beings and is of a pleasant appearance, odour and feel.

The compositions according to the invention are generally in the form of compositions of the type of direct emulsions obtained by dispersion of a fatty phase in an aqueous phase (O/W), inverse emulsions obtained by dispersing an aqueous phase in a fatty phase (W/O) or multiple emulsions obtained by dispersing an inverse emulsion in an aqueous phase (W/O/W). These compositions are prepared according to the usual methods.

More particularly it is direct emulsions obtained by dispersion of a fatty phase in an aqueous phase (O/W).

According to one exemplary embodiment, the emulsions in the kit in accordance with the invention have a liquid fatty phase comprising at least one oil.

In both cases, compounds X and Y are advantageously present in the oily phase.

As examples of oils for use in the composition according to the invention, we may mention:
hydrocarbon oils of animal origin, such as perhydrosqualene;
hydrocarbon oils of vegetable origin, such as liquid triglycerides of fatty acids having from 4 to 10 carbon atoms such as triglycerides of heptanoic or octanoic acids or alternatively, for example sunflower oil, maize oil, soya oil, cucurbit oil, grapeseed oil, sesame oil, hazelnut oil, apricot oil, macadamia oil, arara oil, castor oil, avocado oil, triglycerides of caprylic/capric acids such as those sold by the company Stearineries Dubois or those sold under the designations Miglyol 810, 812 and 818 by the company Dynamit Nobel, jojoba oil, shea butter oil;
synthetic esters and ethers, notably of fatty acids, such as the oils of formulae R₁COOR₂ and R₁OR₂ in which R₁ represents the residue of a fatty acid having from 8 to 29 carbon atoms, and R₂ represents a linear or branched hydrocarbon chain, containing from 3 to 30 carbon atoms, for example purcelline oil, isononyl isononanoate, isopropyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearyl malate, triisocetyl citrate, heptanoates, octanoates, decanoates of fatty alcohols; polyol esters, such as propylene glycol dioctanoate, neopentylglycol diheptanoate and diethylene glycol diisononanoate; and esters of pentaerythritol such as pentaerythrityl tetraisostearate;
linear or branched hydrocarbons, of mineral or synthetic origin, such as volatile or non-volatile paraffin oils, and derivatives thereof, isohexadecane, isododecane, petroleum jelly, polydecenes, hydrogenated polyisobutene such as Parleam® oil;
natural or synthetic essential oils such as, for example, eucalyptus oil, hybrid lavender oil, lavender oil, vetiver oil, litsea cubeba oil, lemon oil, sandalwood oil, rosemary oil, chamomile oil, savory oil, nutmeg oil, cinnamon oil, hyssop oil, caraway oil, orange oil, geraniol, cade oil and bergamot oil;
fatty alcohols having from 8 to 26 carbon atoms, such as cetyl alcohol, stearyl alcohol and their mixture (cetylstearyl alcohol), octyl dodecanol, 2-butyloctanol, 2-hexyldecanol, 2-undecylpentadecanol, oleic alcohol or linoleic alcohol;
fluorinated oils, partially. hydrocarbon-containing and/or silicone-containing, such as those described in document JP-A-2-295912;
silicone oils such as volatile or non-volatile polymethylsiloxanes (PDMS) with linear or cyclic silicone chain, liquid or pasty at room temperature, notably cyclopolydimethylsiloxanes (cyclomethicones) such as cyclohexasiloxane and cyclopentasiloxane; polydimethylsiloxanes bearing alkyl, alkoxy or phenyl groups, pendant or at the end of the silicone chain, groups having from 2 to 24 carbon atoms; phenylated silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes;
mixtures thereof.

In the list of oils given above, “hydrocarbon oil” means any oil comprising mainly carbon atoms and hydrogen atoms, and optionally ester groups, ether groups, fluorine, carboxylic acid and/or alcohol.

The other fats that can be present in the oily phase are for example fatty acids having from 8 to 30 carbon atoms, such as stearic acid, lauric acid, palmitic acid and oleic acid; waxes such as lanolin, beeswax, carnauba wax or candelilla wax, paraffin wax, lignite wax or microcrystalline waxes, ceresine or ozokerite, synthetic waxes such as polyethylene waxes, Fischer-Tropsch waxes; gums such as silicone gums (dimethiconol); silicone resins such as trifluoromethyl C1-4-alkyl dimethicone and trifluoropropyl dimethicone.

These fats can be selected variously by a person skilled in the art in order to prepare compositions having desired properties, for example of consistency or texture.

The compositions according to the invention can additionally contain a volatile oil.

By “volatile oil”, we mean in the sense of the invention an oil that can evaporate in contact with keratinous substances in less than an hour, at room temperature and atmospheric pressure. The volatile organic solvents(s) and the volatile oils of the invention are organic solvents and volatile cosmetic oils, liquid at room temperature, having a non-zero vapour pressure, at room temperature and atmospheric pressure, ranging in particular from 0.13 Pa to 40 000 Pa (10⁻³ to 300 mmHg), in particular in the range from 1.3 Pa to 13 000 Pa (0.01 to 100 mmHg), and more particularly in the range from 1.3 Pa to 1300 Pa (0.01 to 10 mmHg).

As volatile oils, we may mention, among others, the cyclic or linear silicones containing from 2 to 6 silicon atoms, such as cyclohexasiloxane, dodecamethylpentasiloxane, decamethyltetrasiloxane, butyltrisiloxane and ethyltrisiloxane. It is also possible to use branched hydrocarbons, for example isododecane, as well as volatile perfluoroalkanes such as dodecafluoropentane and tetradecafluorohexane, sold under the designations “PF 5050®” and “PF 5060®” by the company 3M and the derivatives of perfluoromorpholine, such as 4-trifluoromethyl perfluoromorpholine sold under the designation “PF 5052®” by the company 3M.

The amount of oily phase present in the compositions according to the invention may range, for example, from 1% to 90% by weight and preferably from 5% to 70% by weight relative to the total weight of the composition under consideration.

The content of volatile oil present in the compositions according to the invention may be, for example, less than or equal to 50% by weight, preferably less than or equal to 30% by weight and better still less than or equal to 10% by weight relative to the total weight of the composition under consideration.

According to an exemplary embodiment, the compositions in accordance with the invention are free of volatile oil.

The emulsions generally contain at least one emulsifier chosen from amphoteric, anionic, cationic and nonionic emulsifiers, used alone or as a mixture. The emulsifiers are generally present in the composition in a proportion that may range, for example, from 0.3% to 30% by weight and preferably from 0.5% to 20% by weight relative to the total weight of the composition.

Needless to say, the emulsifying system is chosen so as to efficiently stabilize the emulsions more particularly under consideration according to the invention, namely those of O/W, W/O or O/W/O. This choice falls within the competence of a person skilled in the art.

Surfactants

For O/W emulsions, we may mention for example as emulsifiers, nonionic surfactants, notably the esters of polyols and of fatty acid with a saturated or unsaturated chain having for example from 8 to 24 carbon atoms and preferably from 12 to 22 carbon atoms, and oxyalkylenated derivatives thereof, i.e. containing oxyethylenated and/or oxypropylenated units, such as the esters of glyceryl and of C₈-C₂₄ fatty acid, and oxyalkylenated derivatives thereof; the esters of polyethylene glycol and of C₈-C₂₄ fatty acid, and oxyalkylenated derivatives thereof; the esters of sorbitol and of C₈-C₂₄ fatty acid, and oxyalkylenated derivatives thereof; the esters of sugar (sucrose, glucose, alkylglucose) and of C₈-C₂₄ fatty acid, and oxyalkylenated derivatives thereof; the ethers of fatty alcohols; the ethers of sugar and of C₈-C₂₄ fatty alcohols, and mixtures thereof

According to one exemplary embodiment, the compositions in accordance with the invention contain, respectively, at least one nonionic surfactant, chosen for example from esters of polyols and of a fatty acid containing a saturated or unsaturated chain, and oxyalkylenated derivatives thereof.

As ester of glyceryl and of fatty acid, we may notably mention glyceryl stearate (mono-, di- and/or tri-glyceryl stearate) (CTFA name: glyceryl stearate) or glyceryl ricinoleate, and mixtures thereof.

As ester of polyethylene glycol and of fatty acid, we may notably mention polyethylene glycol stearate (mono-, di- and/or tri-stearate of polyethylene glycol), and more especially polyethylene glycol monostearate 50 OE (CTFA name: PEG-50 stearate), polyethylene glycol monostearate 100 OE (CTFA name: PEG-100 stearate) and mixtures thereof.

It is also possible to use mixtures of these surfactants, for example the product containing glyceryl stearate and PEG-100 stearate, marketed under the designation ARLACEL 165 by the company Uniqema, and the product containing glyceryl stearate (mono-diglyceryl stearate) and potassium stearate, marketed under the designation TEGIN by the company Goldschmidt (CTFA name: glyceryl stearate SE).

As ester of fatty acid and of glucose or of alkylglucose, we may mention in particular glucose palmitate, alkylglucose sesquistearates such as methylglucose sesquistearate, alkylglucose palmitates such as methylglucose palmitate or ethylglucose palmitate, aliphatic esters of methylglucoside and more especially the diester of methylglucoside and oleic acid (CTFA name: methyl glucose dioleate); mixed ester of methylglucoside and of oleic acid/hydroxystearic acid mixture (CTFA name: methyl glucose dioleate/hydroxystearate); ester of methylglucoside and isostearic acid (CTFA name: methyl glucose isostearate); ester of methylglucoside and lauric acid (CTFA name: methyl glucose laurate); mixture of monoester and diester of methylglucoside and isostearic acid (CTFA name: methyl glucose sesqui-isostearate); mixture of monoester and diester of methylglucoside and stearic acid (CTFA name: methyl glucose sesquistearate) and in particular the product marketed under the designation Glucate SS by the company AMERCHOL, and mixtures thereof.

As oxyethylenated ethers of fatty acid and of glucose or of alkylglucose, we may mention for example the oxyethylenated ethers of fatty acid and of methylglucose, and in particular the polyethylene glycol ether of diester of methyl glucose and stearic acid with about 20 moles of ethylene oxide (CTFA name: PEG-20 methyl glucose distearate) such as the product marketed under the designation Glucam E-20 distearate by the company AMERCHOL; the polyethylene glycol ether of the mixture of monoester and diester of methyl glucose and stearic acid with about 20 moles of ethylene oxide (CTFA name: PEG-20 methyl glucose sesquistearate) and in particular the product marketed under the designation Glucamate SSE-20 by the company AMERCHOL and that marketed under the designation Grillocose PSE-20 by the company GOLDSCHMIDT, and mixtures thereof.

As esters of sucrose, we may mention for example sucrose palmito-stearate, sucrose stearate and sucrose monolaurate.

As ethers of fatty alcohols, we may mention for example the ethers of polyethylene glycol and of fatty alcohol having from 8 to 30 carbon atoms, and notably from 10 to 22 carbon atoms, such as the ethers of polyethylene glycol and cetyl alcohol, stearyl alcohol, cetearyl alcohol (mixture of cetyl and stearyl alcohols). We may mention for example the ethers having from 1 to 200 and preferably from 2 to 100 oxyethylenated groups, such as those of CTFA name Ceteareth-20, Ceteareth-30, and mixtures thereof.

As ethers of sugar, we may notably mention the alkylpolyglucosides, and for example decylglucoside such as the product marketed under the designation MYDOL 10 by the company Kao Chemicals, the product marketed under the designation PLANTAREN 2000 by the company Henkel, and the product marketed under the designation ORAMIX NS 10 by the company Seppic; caprylyl/capryl glucoside such as the product marketed under the designation ORAMIX CG 110 by the Company Seppic or under the designation LUTENSOL GD 70 by the Company BASF; laurylglucoside such as the products marketed under the designations PLANTAREN 1200 N and PLANTACARE 1200 by the company Henkel; coco-glucoside such as the product marketed under the designation PLANTACARE 818/UP by the company Henkel; cetostearyl glucoside optionally mixed with cetostearyl alcohol, marketed for example under the designation MONTANOV 68 by the company Seppic, under the designation TEGO-CARE CG90 by the company Goldschmidt and under the designation EMULGADE KE3302 by the company Henkel; arachidyl glucoside, for example in the form of the mixture of arachidic and behenic alcohols and of arachidyl glucoside marketed under the designation MONTANOV 202 by the company Seppic; cocoylethylglucoside, for example in the form of mixture (35/65) with cetyl and stearyl alcohols, marketed under the designation MONTANOV 82 by the company Seppic, and mixtures thereof.

For the W/O emulsions, examples of emulsifiers that may be mentioned include dimethicone copolyols such as the mixture of cyclomethicone and of dimethicone copolyol, sold under the name DC 5225 C by the company Dow Corning, and alkyl dimethicone copolyols such as the lauryl methicone copolyol sold under the name Dow Corning 5200 Formulation Aid by the company Dow Corning and the cetyl dimethicone copolyol sold under the name Abil EM 90R by the company Goldschmidt, or the polyglyceryl-4 isostearate/cetyl dimethicone copolyol/hexyl laurate mixture sold under the name Abil WE 09 by the company Goldschmidt. One or more coemulsifiers may also be added thereto. Advantageously, the coemulsifier may be chosen from the group comprising polyol alkyl esters. Polyol alkyl esters that may especially be mentioned include glycerol and/or sorbitan esters, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

A crosslinked solid elastomeric organopolysiloxane comprising at least one oxyalkylene group, such as those obtained according to the procedure of examples 3, 4 and 8 of document U.S. Pat. No. 5,412,004 and of the examples of document U.S. Pat. No. 5,811,487, especially the product of example 3 (example of synthesis) of patent U.S. Pat. No. 5,412,004, and such as the product sold under the reference KSG 21 by the company Shin-Etsu, may also be used as W/O emulsion surfactant.

As emulsifier suitable for obtaining a W/O emulsion, polyisobutylene surfactants containing esterified succinic end groups are especially suitable for use, such as those sold under the names Lubrizol 5603® and Chemcinnate 2000® by the companies Lubrizol and Chemron.

### Amphiphilic Polymers

The term “amphiphilic polymer” means any polymer comprising both a hydrophilic portion and a hydrophobic portion and having the property of forming a film that separates two liquids of different polarity and thus making it possible to stabilize liquid-liquid dispersions of direct, inverse or multiple type. The amphiphilic polymers that are more particularly suitable reduce the water/oil interface tension to 10 mN/m, irrespective of the oil. These polymers are ionic (anionic or cationic) or amphoteric. They may be water-soluble or water-dispersible. The term water-soluble means that they can be dispersed in water in the form of a molecular solution. The term water-dispersible means that they can be dispersed in water in particulate form.

The amphiphilic polymers that are suitable for use in the invention generally have a number-average molecular weight ranging from 1000 to 20 000 000 g/mol, preferably ranging from 20 000 to 8 000 000 and even more preferentially from 100 000 to 700 000 g/mol. The amounts of amphiphilic polymers used according to the invention will be chosen from 0.01% to 20%, preferably from 0.1% to 10% and even more preferentially from 0.2% to 5% by weight, relative to the total weight of the composition containing it.

Acrylate/C₁₀-C₃₀-alkylacrylate copolymers such as the products sold under the names Pemulen TR1®, Pemulen TR2® and Carbopol 1382® by the company Goodrich, or mixtures thereof, may be used more particularly. The acrylate/steareth-20 itaconate copolymers and acrylate/ceteth-20 itaconate copolymers sold under the names Structure 2001® and Structure 3001® by the company National Starch may also be used. As terpolymers that may be used, mention may be made of the methacrylic acid/methyl acrylate/behenyl dimethyl m-isopropenylbenzylisocyanate terpolymer ethoxylated with 40 OE, i.e. comprising 40 oxyethylene groups, sold by the company Amerchol under the name Viscophobe DB 1000 NP3-NP4.

Mention may also be made of crosslinked terpolymers of methacrylic acid, of ethyl acrylate and of polyethylene glycol (10 OE) stearyl ether (Steareth 10), especially those sold by the company Allied Colloids under the name Salcare SC 80.

The anionic polymers that may be used according to the invention are, for example, isophthalic acid or sulfoisophthalic polymers, and in particular the phthalate/sulfoisophthalate/glycol copolymers (for example diethylene glycol/phthalate/isophthalate/1,4-cyclohexanedimethanol) sold under the names Eastman AQ Polymer (AQ35S, AQ38S, AQ55S, AQ48 Ultra) by the company Eastman Chemical.

The compositions according to the invention may also comprise at least one dyestuff chosen, for example, from pigments, nacres, dyes and materials with an effect, and mixtures thereof.

These dyestuffs may be present in a content ranging from 0.01% to 50% by weight and preferably from 0.01% to 30% by weight relative to the total weight of the composition.

The compositions according to the invention may comprise a filler especially in a content ranging from 0.01% to 50% by weight and preferably ranging from 0.01% to 30% by weight relative to the total weight of the composition. These fillers may be mineral or organic of any shape, platelet-shaped, spherical or oblong, irrespective of the crystallographic form (for example lamellar, cubic, hexagonal, orthorhombic, etc.). Mention may be made of talc, mica, kaolin, lauroyllysine, starch, boron nitride, barium sulfate, precipitated calcium carbonate, magnesium carbonate, magnesium hydrogen carbonate, hydroxyapatite, glass or ceramic microcapsules, and metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate.

The composition according to the invention may also contain various adjuvants commonly used in cosmetics, such as sequestrants; fragrances; and thickeners and gelling agents. The amounts of these various adjuvants and the nature thereof will be chosen so as not to harm the optical properties of the composition.

Depending on the fluidity of the composition that is to be produced, one or more gelling agents, notably hydrophilic, i.e. soluble or dispersible in water, can be incorporated in the composition.

As hydrophilic gelling agents, we may mention in particular water-soluble or water-dispersible thickening polymers. The latter can notably be selected from: modified or unmodified carboxyvinyl polymers, such as the products marketed under the designations Carbopol® (CTFA name: carbomer) by the company Noveon; polyacrylates and polymethacrylates such as the products sold under the designations Lubrajel® and Norgel® by the company GUARDIAN or under the designation Hispagel® by the company HISPANO CHIMICA; polyacrylamides; polymers and copolymers of 2-acrylamido 2-methylpropane sulphonic acid, optionally crosslinked and/or neutralized, such as poly(2-acrylamido 2-methylpropane sulphonic acid) marketed by the company CLARIANT under the designation Hostacerin AMPS® (CTFA name: ammonium polyacryldimethyltauramide); crosslinked anionic copolymers of acrylamide and of AMPS, in the form of a W/O emulsion, such as those marketed under the name of SEPIGEL 305® (CTFA name: Polyacrylamide/C₁₃₋₁₄ Isoparaffin/Laureth-7) and under the name of SIMULGEL 600® (CTFA name: Acrylamide/Sodium acryloyldimethyltaurate copolymer/Isohexadecane/Polysorbate 80) by the company SEPPIC; polysaccharide biopolymers such as xanthan gum, guar gum, carob gum, acacia gum, scleroglucans, derivatives of chitin and of chitosan, carrageenans, gellans, alginates, celluloses such as microcrystalline cellulose, carboxymethylcellulose, hydroxymethylcellullose and hydroxypropylcellulose; and mixtures thereof.

As lipophilic gelling agents, we may mention for example modified clays such as modified magnesium silicate (bentone gel VS38® from RHEOX), hectorite modified with distearyl dimethyl ammonium chloride (CTFA name: disteardimonium hectorite) marketed under the designation bentone 38 CE® by the company RHEOX.

For application in particular for the care or for make-up of greasy skin, the composition according to the invention can include at least one active selected from: desquamating agents, anti-seborrhoeic agents, antimicrobial agents, and soothing agents.

For application in particular for the care or make-up of aged skin, the composition according to the invention can include at least one active selected from: desquamating or hydrating agents; depigmenting or anti-pigmenting agents; antiglycating agents; anti-NO agents; agents stimulating the synthesis of dermal or epidermal macromolecules and/or preventing their degradation; agents stimulating the proliferation of fibroblasts or of keratinocytes and/or differentiation of keratinocytes; muscle relaxants or dermo-decontracting agents; anti-radical or anti-pollution agents; lifting agents; and agents acting on the microcirculation.

The products and compositions according to the invention can be in the form of a care or make-up product for the face and/or the body, and can be packaged for example in the form of cream in a pot or of fluid in a tube or in a pump bottle.

They may advantageously be used for caring for and/or making up the skin, the lips, keratin fibres and in particular the eyelashes and/or the nails, depending on the nature of the ingredients used.

According to one embodiment, the compositions are compositions for making up and/or caring for the lips.

In particular, the kit in accordance with the invention may be intended to afford a film for coating bodily or facial skin.

In particular, the kit in accordance with the invention may be intended to affording a film for coating the lips or the nails.

According to another embodiment, the kit in accordance with the invention may be intended to afford a film for coating keratin fibres.

According to another embodiment, the compositions are compositions for coating the eyelashes or the eyebrows and more particularly mascaras.

According to another embodiment, the compositions are compositons for coating the skin of the body or of the face, and more particularly compositions for make-up and/or care of the skin of the body or of the face such as, for example, foundations or compositions for make-up of the body.

A person skilled in the art will be able to select the appropriate galenical form, as well as its method of preparation, on the basis of his general knowledge, taking into account, on the one hand, the nature of the constituents used, notably their solubility in the carrier, and, on the other hand, the application envisaged for each composition.

The invention is illustrated in more detail by the examples described below. Unless stated otherwise, the quantities shown are expressed as percentage by weight.

### EXAMPLES

In the examples of compositions described hereunder, the combination of the following mixtures A and B produced by the company Dow Corning is used as combination of the compounds X and Y:

### Mixture A:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| 1,3-Diethenyl-1,1,3,3- | 68478-92-2 | Trace | Catalyst |
| Tetramethyldisiloxane | | | |
| complexes | | | |
| Tetramethyldivinyldisiloxane | 2627-95-4 | 0.1-1 | Polymer |

### Mixture B:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| Dimethyl, | 68037-59-2 | 1-10 | Polymer |
| Methylhydrogen | | | |
| Siloxane, trimethylsiloxy- | | | |
| terminal | | | |

### Example 1

Cosmetic Composition Containing a Compound A in Accordance with the Invention in the Form of an Oil-in-Water Emulsion

| | |
|---|---|
| Phase A | |
| Mixture of glyceryl stearate and PEG-100 stearate | 2.00 g |
| (Arlacel ® 165 FL from Uniqema) | |
| Mixture of (C₁₄-C₁₅ linear) dialkyl tartrate, cetylstearyl | 1.50 g |
| alcohol and oxyethylenated (25 OE) oxypropylenated | |
| (25 OP) lauryl alcohol (Cosmacol PSE from Sasol) | |
| Cyclohexasiloxane | 10.00 g |
| Stearyl alcohol | 1.00 g |
| Mixture A | 10.00 g |
| Phase B | |
| Water | qs |
| Phenoxyethanol | 1.00 g |
| Pentasodium salt of ethylenediaminetetramethylenephosphonic | 0.05 g |
| acid | |
| Polyacrylamidomethylpropanesulfonic acid partially | 0.40 g |
| neutralized with aqueous ammonia and highly crosslinked | |
| (Hostacerin AMPS from Clariant) | |
| Xanthan gum | 0.20 g |

### Example 2

### Cosmetic Composition Containing a Compound B Complementary to Compound 1 of Example 1 in the Form of an Oil-in-Water Emulsion

| | |
|---|---|
| Phase A | |
| Mixture of glyceryl stearate and PEG-100 stearate | 2.00 g |
| (Arlacel ® 165 FL from Uniqema) | |
| Mixture of (C₁₄-C₁₅ linear) dialkyl tartrate, | 1.50 g |
| cetylstearyl alcohol and oxyethylenated (25 OE) | |
| oxypropylenated (25 OP) lauryl alcohol | |
| (Cosmacol PSE from Sasol) | |
| Cyclohexasiloxane | 10.00 g |
| Stearyl alcohol | 1.00 g |
| Mixture B | 10.00 g |
| Phase B | |
| Water | qs |
| Phenoxyethanol | 1.00 g |
| Pentasodium salt of ethylenediaminetetramethylenephosphonic | 0.05 g |
| acid | |
| Polyacrylamidomethylpropanesulfonic acid partially | 0.40 g |
| neutralized with aqueous ammonia and highly | |
| crosslinked (Hostacerin AMPS from Clariant) | |
| Xanthan gum | 0.20 g |

### Procedure for Examples 1 and 2:

Phase B is heated to about 75° C. and the Hostacerin AMPS is incorporated therein with stirring until a uniform gel is obtained.

Phase A is then heated to about 75° C.

The emulsion is prepared by incorporating phase A into phase B and stirring until the composition has completely cooled to room temperature.

Demonstration of the Matting Properties of a Film Obtained from the Mixture of the Compositions of Examples 1 and 2

The compositions I and II in emulsion form described above (examples 1 and 2) are mixed together in a 50/50 weight proportion and the mixture is then applied to a contrast card (Prufkarte type 24/5-250 cm² sold by the company Erichsen) using a mechanical film spreader (wet thickness 150 microns). The composition is dried overnight at a temperature of 37° C. A matt film that does not transfer is obtained.

The result obtained is the ratio R between the specular reflection and the diff-use reflection. The value of R is proportionately smaller the greater the matting effect.

The respective mattnesses of compositions I and II are also measured. The results are given in the table below.

| | Composition | | |
|---|---|---|---|
| | I | II | Mixture I + II |
| R | 1.45 ± 0.09 | 1.45 ± 0.10 | 0.66 ± 0.03 |

These in vitro results show that compositions I and II of the invention, once mixed together, spread and dried, form a deposit that has good matting properties. The value is markedly lower than that expected with a mixture of reactive silicones in two oily phases at the same concentration as that formulated in the oily phase of the emulsion is in accordance with the invention.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Cosmetic kit for make-up or non therapeutic care of keratinous substance(s) comprising at least two compositions that are different and are packaged separately, the kit comprising at least one compound X, at least one compound Y, and at least one catalyst, with at least one of compounds X or Y being a silicone compound and said compounds X and Y being capable of reacting together by a hydrosilylation reaction in the presence of a catalyst, when they are brought into contact with one another, said kit being such that compounds X, Y and the catalyst are not present simultaneously in the same composition and that at least one of the compositions of said kit is in the form of a simple or multiple W/O/W emulsion in which said compound X or Y is present in the oily phase.

2. Kit according to claim 1 comprising at least:
i. a first composition containing in a physiologically acceptable medium at least one compound X and
ii. a second composition containing in a physiologically acceptable medium at least one compound Y,
with at least one of said first and second compositions being in the form of a simple or multiple W/O/W emulsion in which the compound X or Y is present in the oily phase, and with at least one of said first and second composition additionally containing at least one catalyst.

3. Kit according to claim 1, in which only one of the first and second compositions is in the form of an emulsion.

4. Kit according to claim 1, in which the first and second compositions are in the form of an emulsion.

5. Kit according to claim 1, comprising at least:
i. a first composition containing, in a physiologically acceptable medium, at least one compound X and one compound Y, said compounds X and Y being capable of reacting together in the presence of a catalyst via a hydrosilylation reaction, when they are placed in contact with each other, and
ii. a second composition containing, in a physiologically acceptable medium, at least said catalyst necessary for the interaction of said compounds X and Y, with said first composition being in the form of a simple or multiple (W/O/W) emulsion in which compounds X and Y are present in the oily phase.

6. Kit according to claim 1, in which the emulsion(s) is (are) direct emulsions (O/W).

7. Kit according to claim 1, in which the emulsions have a liquid fatty phase comprising at least one oil.

8. Kit according to claim 7, in which the oil or oils are present at a content ranging from 1 to 90 wt. % relative to the total weight of each composition.

9. Kit according to claim 1, in which the content of volatile oil is less than or equal to 50% by weight relative to the total weight of each composition.

10. Kit according to claim 1, in which the compositions are free of volatile oil.

11. Kit according to claim 1, in which the compositions contain, respectively, at least one nonionic surfactant.

12. Kit according to claim 11, in which the nonionic surfactant is chosen from esters of polyols and of a fatty acid containing a saturated or unsaturated chain, and oxyalkylenated derivatives thereof.

13. Kit according to claim 12, in which the nonionic surfactant is chosen from glyceryl esters of a C₈-C₂₄ fatty acid, polyethylene glycol esters of a C₈-C₂₄ fatty acid, sorbitol esters of a C₈-C₂₄ fatty acid and sugar esters of a fatty acid, and oxyalkylenated derivatives thereof; fatty alcohol ethers; ethers of sugar and of fatty alcohols, and mixtures thereof.

14. Kit according to claim 1, wherein compound X is selected from silicone compounds comprising at least two unsaturated aliphatic groups.

15. Kit according to the claim 14, in which the compound X is a polyorganosiloxane comprising a silicone main chain whose unsaturated aliphatic groups are pendant from the main chain (side group) or located at the ends of the main chain of the compound (end group).

16. Kit according to the claim 15, wherein compound X bears at least one polar group.

17. Kit according to claim 1, wherein compound X is selected from the polyorganosiloxanes comprising at least two unsaturated aliphatic groups each attached to a silicon atom.

18. Kit according to claim 1, wherein compound X is selected from the polyorganosiloxanes containing siloxane units of formula: 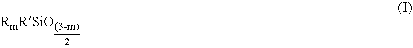
in which:
R represents a linear or cyclic, monovalent hydrocarbon group, having from 1 to 30 carbon atoms,
m is equal to 1 or 2 and
R′ represents:
an unsaturated aliphatic hydrocarbon group having from 2 to 10 carbon atoms or
an unsaturated cyclic hydrocarbon group having from 5 to 8 carbon atoms.

19. Kit according to claim 18, in which the polyorganosiloxane of formula (I) is such that R′ represents a vinyl group or a group —R″—CH═CHR′″ in which R″ is a divalent aliphatic hydrocarbon chain, having from 1 to 8 carbon atoms, bound to the silicon atom and R′″ is a hydrogen atom or an alkyl radical having from 1 to 4 carbon atoms.

20. Kit according to claim 18, wherein R represents an alkyl radical having from 1 to 10 carbon atoms or alternatively a phenyl group, and R′ is a vinyl group.

21. Kit according to claim 15, wherein the polyorganosiloxanes additionally comprise units of formula: 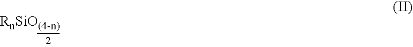
in which R represents a linear or cyclic, monovalent hydrocarbon group, having from 1 to 30 carbon atoms, and n is equal to 1, 2 or 3.

22. Kit according to claim 1, wherein compound X is selected from organic oligomers or polymers, hybrid organic/silicone oligomers or polymers, said oligomers or polymers bearing at least 2 unsaturated reactive aliphatic groups.

23. Kit according to claim 1 in which compound Y comprises at least two free Si—H groups.

24. Kit according to claim 1, wherein compound Y is selected from the polyorganosiloxanes comprising at least one alkylhydrogenosiloxane unit with the following formula: 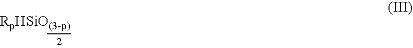
in which:
R represents a linear or cyclic, monovalent hydrocarbon group, having from 1 to 30 carbon atoms or a phenyl group, and p is equal to 1 or 2.

25. Kit according to claim 24, in which the compound Y is such that the radicals R represent a C₁-C₁₀ alkyl group.

26. Kit according to claim 23, in which Y is a polyorganosiloxane comprising at least two alkylhydrogenosiloxane units of formula —(H₃C)(H)Si—O—.

27. Kit according to claim 1, in which the catalyst is a catalyst based on platinum or tin.

28. Kit according to the claim 27, wherein the catalyst is present in a content ranging from 0.0001 to 20 wt. % relative to the total weight of the composition containing it.

29. Kit according to claim 1, wherein compound X is a polydimethylsiloxane with vinylic end groups and compound Y is a polymethylhydrogenosiloxane.

30. Kit according to claim 1, in which compound X bears at least one polar group that is able to form a hydrogen bond with keratinous substances.

31. Kit according to claim 1, comprising, in at least one of the compositions, a filler selected from silica or surface-treated silica.

32. Kit according to claim 1, wherein compound X has a weight-average molecular weight (Mw) in the range from 150 to 1 000 000.

33. Kit according to claim 1, wherein compound Y has a weight-average molecular weight (Mw) in the range from 200 to 1 000 000.

34. Kit according to claim 1, wherein compound X represents from 0.1 to 95 wt. % relative to the total weight of the composition containing it.

35. Kit according to claim 1, wherein compound Y represents from 0.1 to 95 wt. % relative to the total weight of the composition containing it.

36. Kit according to claim 1, wherein compounds X and Y are present in a molar ratio X/Y in the range from 0.05 to 20.

37. Kit according to claim 1, in which at least one of the compositions further comprises at least one colorant.

38. Kit according to claim 1, in which at least one of the compositions comprises at least one filler.

39. Kit according to claim 1, in which the compositions are packaged separately in the same packaging article.

40. Kit according to claim 1, for affording a film for coating bodily or facial skin.

41. Kit according to claim 1, for affording a film for coating the lips.

42. Kit according to claims 1, for affording a film for coating the nails.

43. Kit according to claims 1, for affording a film for coating keratin fibres.

44. Method of cosmetic care and/or of make-up of keratinous substance(s) comprising at least the application in the form of at least a simple or multiple (W/O/W) emulsion (a), of one or more compounds X (b), of one or more compounds Y with at least one of compounds X and Y being a silicone compound and said compounds X and Y being capable of reacting together by a hydrosilylation reaction in the presence of a catalyst, when they are brought into contact with one another, and (c) of a catalyst necessary for the interaction of said compound X with said compound Y and applications (a), (b) and (c) can be simultaneous or consecutive in any order provided that it promotes the interaction of said compounds X and Y.

45. Method according to claim 44, comprising applying, on said keratinous substances, at least one composition in the form of a simple or multiple W/O/W emulsion comprising, in its oily phase, at least one compound X, at least one compound Y, said composition containing additionally at least one catalyst.

46. Method according to claim 44, in which the composition is obtained by mixing, at the time of use, at least one first composition comprising at least compound X and a second composition comprising at least compound Y, at least one of the first and second compositions being in the form of a simple or multiple W/O/W emulsion, and at least one of said first and second composition additionally comprising at least one catalyst.

47. Cosmetic method for make-up and/or care of keratinous substances, comprising the application on said keratinous substances:
(i) of at least one layer of a first composition containing in a physiologically acceptable medium at least one compound X;
(ii) of at least one layer of a second composition containing in a physiologically acceptable medium at least one compound Y,
at least one of compounds X and Y being a silicone compound, said compounds X and Y being capable of reacting together by a hydrosilylation reaction in the presence of a catalyst, when they are brought into contact with one another, with at least one of said first and second compositions being in the form of a simple or multiple W/O/W emulsion comprising compound X or Y in its oily phase, and with at least one of said first and second composition additionally containing at least one catalyst.

48. Method according to claim 44 or 47, in which the composition(s) are as defined in claim 1.

49. Cosmetic kit for make-up or non therapeutic care of keratinous substance(s) comprising at least two compositions that are different and are packaged separately, the kit comprising at least one compound X, at least one compound Y, and optionally at least one catalyst, with at least one of compounds X or Y being a silicone compound and said compounds X and Y being capable of reacting together by a condensation reaction, if necessary in the presence of a catalyst, when they are brought into contact with one another, said kit being such that compounds X and Y and the catalyst, when it is present, are not present simultaneously in the same composition and that at least one of the compositions of said kit is in the form of a simple or multiple W/O/W emulsion in which said compound X or Y is present in the oily phase.
